(19) United States
(12) Patent Application Publication
(10) Pub. No.: US 2026/0009017 A1
(43) Pub. Date: Jan. 8, 2026

(54) TISSUE DISSOCIATION DEVICE

(71) Applicants: The Broad Institute, Inc., Cambridge, MA (US); The General Hospital Corporation, Boston, MA (US); President and Fellows of Harvard College, Cambridge, MA (US)

(72) Inventors: Michael KIM, Cambridge, MA (US); Evan MACOSKO, Boston, MA (US); Naeem NADAF, Cambridge, MA (US)

(73) Assignees: The Broad Institute, Inc., Cambridge, MA (US); The General Hospital Corporation, Boston, MA (US); President and Fellows of Harvard College, Cambridge, MA (US)

(21) Appl. No.: 19/333,769

(22) Filed: Sep. 19, 2025

Related U.S. Application Data
(63) Continuation of application No. PCT/US2024/021205, filed on Mar. 22, 2024.
(60) Provisional application No. 63/453,842, filed on Mar. 22, 2023.

(51) Int. Cl. C12N 15/10; G01N 1/28

(52) U.S. Cl. CPC C12N 15/1017; G01N 1/286

(57) ABSTRACT

The technology described herein provides tissue dissociation well plates, devices, systems, and kits to isolate single-cells or a single-nuclei using wells with roughened, angled bottom surfaces to receive pipette tips delivering tissue samples and isolation buffers. In certain examples, the bottom surfaces of the wells are roughened to aid in breaking down the tissue sample. In other examples, the tip of the pipette is roughened or serrated to aid in breaking down the tissue sample. The wells may be arrayed in a solid rigid upper surface. The pipette tips deliver isolation buffers and/or dissociation fluids to the wells and the tissue samples. The fluid delivery is provided by pumps via one or more perfusion manifolds. A pipette adaptor raises, lowers, and twists the pipette tips. The pipette tips deliver the dissociation fluid, withdraw the tissue samples with a suction force, and return the tissue samples to the well with an expelling force. The pipette tips may be twisted to provide an additional force to break down the tissue sample.

### CROSS-REFERENCE TO RELATED APPLICATION

The present application is a Continuation of International Application No. PCT/US2024/021205, filed on Mar. 22, 2024, and is related to and claims priority under 35 U.S.C. § 119(e) to U.S. Provisional Patent Application No. 63/453,842, entitled “TISSUE DISSOCIATION DEVICE,” filed Mar. 22, 2023. The entire contents of the aforementioned patent application is incorporated herein by this reference.

### STATEMENT AS TO FEDERALLY SPONSORED RESEARCH

This invention was made with government support under Grant No. 1U19MH114821 awarded by the National Institutes of Health. The government has certain rights in the invention.

### TECHNICAL FIELD

The present disclosure relates to automatic cell dissociation devices. More particularly, the disclosure relates to automatic cell dissociation devices for providing high-throughput isolation of single-cells or single-nuclei with an increased recovery rate and improved ease-of-use workflow.

### BACKGROUND

In conventional dissociation processes, obtaining single-cells or single-nuclei for sequencing is labor- and time-intensive. For example, a trained lab technician may take up to a week (from initial sample/tissue processing to initial data analysis) to obtain a single cell or single nuclei for a single experiment. Prior art cell/nuclei isolation protocols are complex and require extensive training for lab technicians, which is a costly and slow process. Further, the nature of such prior art cell isolation protocols creates significant problems for comparative analysis of data generated from isolated single cells/nuclei because different batches of isolated single cells/nuclei prepared by different lab technicians (e.g., in the same lab or in different labs) may be prone to batch specific errors.

Conventional cell/nuclei isolation and dissociation processes are also cost-prohibitive as single experiments may cost $10,000 or more. Consequently, batch-dependent errors or low-quality data resulting from poor sample preparation by different technicians wastes valuable research dollars.

Conventional cell/nuclei dissociation devices are not capable of producing quality samples for sequencing for a number of reasons. For example, such devices fail to dissociate samples (e.g., tissue samples) completely and often allow recovery of only low numbers of cells/nuclei from the sample (e.g., tissue). Conventional dissociation devices are also very expensive. Additionally, such devices are not conducive to parallel processing, which greatly slows sample processing speed.

Conventional dissociation devices and processes do not allow automated high-quality high-throughput sample preparation of single-cell and single-nuclei to be run in parallel in a manner that would promote the ability of scientists to scale generation of relevant data sets and to facilitate comparison of results across samples or sample sets. Accordingly, there is an urgent need for dissociation devices and processes that allow scalable high-throughput preparation and analysis of single-cell and/or single-nuclei samples.

### SUMMARY OF THE INVENTION

The present disclosure provides devices and methods for isolating single-cells or a single-nuclei in a high-throughput system that enables parallel processing of a large number of samples (e.g., tissue samples). In exemplary embodiments, the devices and methods herein provide well plates comprising wells with roughened and/or angled bottom surfaces that receive pipette tips that deliver isolation buffers including single-cells or single-nuclei (e.g., tissue samples). The angled bottom surfaces may be between about 10 and 40 degrees, or between about 15 and 30 degrees. In certain examples, the bottom surface of the wells are roughened to aid in breaking down a tissue sample, such as when the pipette tips are twisted. In other examples, the pipette tip may alternatively or additionally be roughened or serrated to aid in breaking down the tissue sample. In certain examples, the well plates may include an array of wells such as, for example, 6, 12, 24, 48, 96, 384, 1536, etc. wells.

The wells may be arrayed in or on a supporting solid surface. The pipette tips are configured to deliver dissociation fluids to the wells and the tissue samples. The fluid delivery may be provided by one or more pumps (e.g., via a perfusion manifold). In some embodiments, a pipette adaptor may be used to raise, lower, and/or twist the pipette tips. The pipette tips are configured to deliver the dissociation fluid, withdraw the tissue samples with a suction force, and return the tissue samples to the well with an expelling force. In exemplary embodiments, the pipette tips may also be twisted to provide an additional force to break down the tissue sample.

In an aspect, the disclosure provides a tissue dissociation plate that includes a rigid upper surface, and a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface.

In embodiments, the angled bottom surface has an angle between 10 degrees and 40 degrees between the lower side or the upper side and the circumferential wall.

In embodiments, the angle is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 degrees.

In embodiments, the angled bottom surface includes a roughened surface internal to each of the plurality of wells.

In embodiments, the roughened surface includes one or more ridges, grooves, scallops, bumps, or pits. In embodiments, the one or more ridges, grooves, or scallops substantially span a horizontal diameter of each of the plurality of wells. In embodiments, the one or more ridges, grooves, or scallops are arrayed in a corresponding number of horizontal rows which are substantially equally spaced apart from the lower side to the upper side of the angled bottom surface. In embodiments, the one or more ridges, grooves, or scallops each include one or more channels which form breaks in each of the corresponding number of horizontal rows. In embodiments, the one or more bumps or pits are substantially evenly spaced across the roughened surface internal to each of the plurality of wells.

In embodiments, the rigid upper surface is substantially flat.

In embodiments, the upper opening has a diameter between 8 mm to 20 mm. In embodiments, the upper opening has a diameter of 8, 9, 10, 11, 12, 13, 14, 15, 16, 17, 18, 19, or 20 mm.

In embodiments, the circumferential wall has a height of 20 mm to 60 mm. In embodiments, the circumferential wall has a height of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mm.

In embodiments, the roughened surface dissociates a tissue sample into single cells or single nuclei by applying a shearing friction to the tissue sample which is introduced via a pipette tip having a distal tip which is not parallel to the angled bottom surface.

In embodiments, the roughened surface dissociates a tissue sample into 121,000 to 5,000,000 single cells or single nuclei per mm³ of the tissue sample. In embodiments, the roughened surface dissociates a tissue sample into 1430,000 to 300,000 single cells or single nuclei per mm³ of the tissue sample.

In embodiments, the roughened surface dissociates a tissue sample into 140,000 to 150,000 single cells or single nuclei per mm³ of the tissue sample.

In an aspect, the disclosure provides a kit to isolate cells and/or single nuclei that includes the above-described tissue dissociation plate, and one or more single cell isolation buffers or single nuclei isolation buffers.

In embodiments, the kit further includes one or more pipette tips each having a distal pipette tip which includes a roughened surface. In embodiments, the roughened surface includes one or more ridges, grooves, scallops, bumps, or pits.

In embodiments, the one or more single cell or nuclei isolation buffers include Buffer 1, Buffer 2 or Buffer 3 where Buffer 1 comprises Na₂SO₄ (5.83 g), K₂SO₄ (2.615 g), Glucose (0.905 g), HEPES (1.2 g), 1M MgCl₂ (2.5 mL) and ddH₂O added to 500 mL, Buffer 2 comprises Buffer 1 (15 mL), 1% Kollidon (0.150 g), 1% TX-100 (150 μl), and 10% BSA (15 μl), and Buffer 3 comprises Nuclei EZ lysis buffer (Sigma N3408-200ML). In embodiments, the one or more single cell or nuclei isolation buffers include Buffer 1 or Buffer 2.

In embodiments, the one or more single cell or nuclei isolation buffers include Buffer 3.

In an aspect, the disclosure provides a tissue dissociation well plate comprising a rigid upper surface and a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface; at least one pump having one or more pump inlets and one or more pump outlets; one or more perfusion manifolds having a manifold inlet in fluid connection with one of the one or more pump outlets and two or more manifold outlets in fluid connection with one or more pipette tips via a corresponding number of fluid tubes; a valve controller configured to regulate fluid flow through the corresponding number of fluid tubes; a manifold configured to reversibly receive the one or more pipette tips on a lower side and the corresponding number of fluid tubes on an upper side, wherein the manifold is configured to raise, lower, and twist the one or more pipette tips; and a controller configured to regulate the pump and cause the manifold to perform a sequence to deliver a fluid to each of the plurality of wells via the one or more pipette tips.

In embodiments, the angled bottom surface has an angle between 10 degrees and 40 degrees between the lower side or the upper side and the circumferential wall. In embodiments, the angled bottom surface includes a roughened surface internal to each of the plurality of wells. In embodiments, the roughened surface includes one or more ridges, grooves, scallops, bumps, or pits. In embodiments, the one or more ridges, grooves, or scallops substantially span a horizontal diameter of each of the plurality of wells. In embodiments, the one or more ridges, grooves, or scallops are arrayed in a corresponding number of horizontal rows which are substantially equally spaced apart from the lower side to the upper side of the angled bottom surface. In embodiments, the one or more ridges, grooves, or scallops each include one or more channels which form breaks in each of the corresponding number of horizontal rows. In embodiments, the one or more bumps or pits are substantially evenly spaced across the roughened surface internal to each of the plurality of wells.

In embodiments, the rigid upper surface is substantially flat.

In embodiments, the upper opening has a diameter between 8 mm to 20 mm, optionally wherein the circumferential wall has a height of 20 mm to 60 mm.

In embodiments, the roughened surface dissociates a tissue sample into single cells or single nuclei by applying a shearing friction to the tissue sample which is introduced via a pipette tip having a distal tip which is not parallel to the angled bottom surface.

In embodiments, the roughened surface dissociates a tissue sample into 125,000 to 160,000 single cells or single nuclei per mm3 of the tissue sample, optionally wherein the roughened surface dissociates a tissue sample into 130,000 to 150,000 single cells or single nuclei per mm³ of the tissue sample.

In embodiments, the tissue dissociation well plate supports an array of 6, 12, 24, 48, 96, 384 or 1536 wells.

In embodiments, the tissue dissociation device further includes an adaptor affixed to the lower side of the manifold configured to reversibly receive the one or more pipette tips.

In embodiments, the adaptor further comprises a motor to twist each of the one or more pipette tips in either a clockwise or counterclockwise direction.

In embodiments, the valve controller comprises multi-tube solenoid based pinch valves to control the flow from the output fluid tubes to the plurality of pipettes.

In embodiments, the sequence to deliver each of the one or more fluids to each of the plurality of wells via the one or more pipettes withdraws tissue samples with a suction on each of the one or more pipettes, lowers each of the one or more pipette tips to form a contact with a bottom surface of each of plurality of wells, injects the tissue samples onto the bottom surface of each of plurality of wells and twists each of the one or more pipettes.

In aspects, the disclosure provides a method to dissociate tissue samples including the steps of: (i) delivering tissue samples to a plurality of wells arrayed on a tissue dissociation well plate, each tissue dissociation well plate comprising a rigid upper surface and a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface; (ii) delivering one or more single cell isolation buffers or single nuclei isolation buffers to each of the plurality of wells via a plurality of pipette tips; (iii) withdrawing, via each of the plurality of pipette tips, the tissue samples from each of the plurality of wells with a suction on each of the plurality of pipette tips; (iv) returning each of the plurality of pipette tips to the bottom of each of the plurality of wells to form a contact with the angled bottom surface of each of the plurality of wells; (v) injecting the tissue samples onto the angled bottom surface of each of the plurality of wells; and (vi) twisting each of the plurality of pipette tips in a circular motion.

In embodiments, the tissue dissociation well plate supports an array of 6, 12, 24, 48, 96, 384 or 1536 wells.

In embodiments, the angled bottom surface includes a roughened surface internal to each of the plurality of wells. In embodiments, the roughened surface includes one or more ridges, grooves, scallops, bumps, or pits. In embodiments, the one or more ridges, grooves, or scallops substantially span a horizontal diameter of each of the plurality of wells and are arrayed in a corresponding number of horizontal rows which are substantially equally spaced apart from a lower end to an upper end of the angled bottom surface. In embodiments, the one or more bumps or pits are substantially evenly spaced across the roughened surface internal to each of the plurality of wells. In embodiments, the roughened surface dissociates a tissue sample into single cells or single nuclei by applying a shearing friction to the tissue sample which is introduced via a pipette tip having a distal tip which is not parallel to the angled bottom surface.

In embodiments, the roughened surface dissociates a tissue sample into 125,000 to 160,000 single cells or single nuclei per mm³ of the tissue sample.

In embodiments, each of the plurality of pipette tips is twisted between 90 degrees and 360 degrees.

In embodiments, the method is repeated one or more times to the tissue samples using one or more different isolation buffers.

In embodiments, steps (i)-(vi) are performed using the tissue dissociation device disclosed herein.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

The drawings are appended hereto and form a portion of this disclosure.

FIGS. 1A-1D illustrate exemplary isolation pipettor systems delivering dissociation fluids to a set of wells. FIG. 1A shows a cross-section view of an exemplary isolation pipettor system including a temperature regulation plate. FIG. 1B shows a cross-section view of an exemplary isolation pipettor system including a final well plate and filters. FIG. 1C shows a cross-section view of an exemplary isolation pipettor system including a dissociation well plate having roughened well systems and/or pipette tips. FIG. 1D shows a cross-section view of an exemplary isolation pipettor system including a temperature regulation plate and wells with angled bottom surfaces.

FIGS. 2A-2D illustrate exemplary embodiments of a pipette tip delivering dissociation fluids to a well with an angled bottom surface. FIG. 2A shows a perspective cross-section view of a well having an angled bottom surface. FIG. 2B shows a perspective cross-section view of a well having an angled bottom surface and including a pipette tip having a roughened tip surface. FIG. 2C shows a perspective cross-section view of a well having an angled bottom surface including a roughened well surface (e.g., a scalloped surface). FIG. 2D shows a front, cross-section view of the wells shown in FIG. 2A and FIG. 2B showing that the angled bottom surface is associated with an angle A and an angle B.

FIGS. 3A-3B illustrate an exemplary embodiment of a well plate and a pipette adaptor reversibly interfaced with an adaptor frame in an exploded view (FIG. 3A) and a non-exploded view (FIG. 3B).

FIGS. 4A-4B illustrate exemplary embodiments of a dissociation device with a pump, manifold, and valve controllers (FIG. 4A) and a pinch valve system (FIG. 4B).

FIG. 5 is an illustration of a dissociation device with 3-way manifolds.

FIG. 6 is a block flow diagram depicting a high-throughput parallel processing titration dissociation method.

FIG. 7 is a block flow diagram depicting a high-throughput optimized dissociation method.

FIG. 8 is an illustration of DAPI peak results from a conventional device and from the dissociation device as described herein.

FIG. 9 is a box and whisker plot of transcripts per nucleus for manual dissociated samples and samples dissociated by the device described herein.

FIG. 10 is a box and whisker plot of genes per nucleus of manual dissociated samples and samples dissociated by the device described herein.

### DETAILED DESCRIPTION

The technology described herein provides dissociation devices to isolate single-cells or a single-nuclei in high-throughput parallel processing procedures to reproducibly produce single cells/nuclei of very high quality. The technology described herein also provides an automated tissue dissociation apparatus, which may include a pump, one or more manifolds, and one or more valves. The technology described herein provides a method to dissociate tissue samples.

The technology described herein provides users an ability to perform single-cell isolation and single-nuclei isolation on multiple concurrent samples for use in, for example, protocols such as those described in Russell et al. (2024) Nature January; 625(7993):101-109. A trained technician using conventional techniques to conduct a single experiment to obtain the single-cell or single-nuclei samples may require up to a week from initial processing to initial data analysis. The technology described herein allows for automated systems that conduct a high number of isolation experiments concurrently, such as 6, 12, 24, 48, 96, 384, or 1536. By performing identical experiments using an automated process that performs the isolation by following regimented and identical methods, the results are repeatable, predictable, and robust.

Data from different batches performed manually by different technicians in different labs may introduce errors when compared to one another. Manual methods that may require multiple weeks to perform and are subject to the training and repeatability of the lab technicians and provide opportunities for variance in the results and contamination of the samples.

Other systems have attempted to automate the process using conventional components and methods. However, these conventional systems have failed to dissociate the tissue samples reliably and completely. These conventional systems have produced low numbers of nuclei as compared to the technology described herein. Further, these conventional systems require a significantly longer processing time because the systems are not conducive to parallel, concurrent processing. Performing the multiple operations concurrently with the technology described herein using a standard automated process creates more repeatable results in a shorter amount of time.

The techniques described herein may use taller or wider wells than a conventional manual system or wells with a greater diameter. The larger wells allow the instant system to minimize froth formation, increase cell recovery, and minimize cross contamination from froth spillover.

The techniques herein further provide devices and methods for regulating temperature during the isolation of single cells or nuclei. For example, the isolation process may occur at temperatures that are cooler than room temperature (e.g., −10° C. to 10° C.) to enhance the ability to isolate single cells and/or single nuclei from tissue samples. In some exemplary embodiments, the temperature may be about 4° C.

The techniques described herein provide a consistently high yield as compared to manual systems. In an example experiment, isolating adult mouse cerebellum nuclei, manual processes produced approximately 120,000 nuclei per mm³ of tissue sample, while the technology using methods described herein recovered approximately 140,000 nuclei per mm³ of tissue sample.

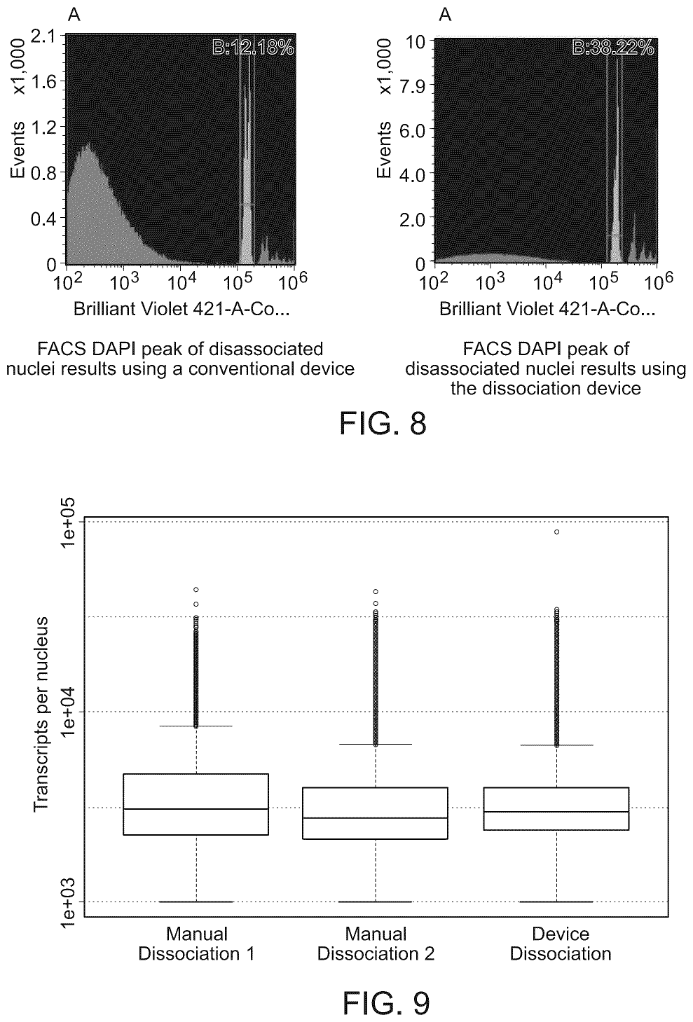

Additionally, conventional processes do not allow sample preparation of single-cell and single-nuclei to be automated. The automated processes herein produce highly reliable results that gives users the ability to compare results across groups, to allow more scientists access to the method, and to meet the increasing demands to process ever-increasing numbers of samples. Further details of the improved yield are discussed with respect to data presented in FIG. 8 through FIG. 10. Using the technology as described herein, the samples are processed more accurately and at a greater rate with lower variability, which greatly reduces the cost per sample.

### Single-Cell Isolation Wells and Kits

Embodiments disclosed herein provide modified dissociation wells for single cell tissue dissociation and recovery, as well as modified tips that may be used with the dissociation wells. The dissociation wells and/or modified tips may be combined together into kits. The kits may further comprise dissociation buffers. As used in the context herein “single-cell tissue dissociation” can refer to both dissociation of single cells from a tissue sample or dissociation of single nuclei from single cells.

A dissociation well can be configured as a single stand-alone well or configured as part of a spaced array, such as on a supporting substrate. The individual wells may be made of standard lab-grade plastics, metals, ceramics, or other suitable materials as known. Likewise, the supporting substrate may be made of standard lab-grade plastics, metals, glass, ceramics, or other suitable materials as known. The dissociation well(s) may have a generally conical shape. The height of the dissociation well may be between about 20 mm and about 60 mm in height, have a diameter at the bottom of the well of about 8 mm to about 20 mm, and a volume of about 2 mL to about 10 mL.

In alternate embodiments, larger wells may be used. For example, in applications in which excess froth is generated in the process, a larger well may be used to keep the froth from spilling out of the wells and contaminating other samples. In these examples, a well may be used that is approximately 250 mm in diameter and approximately 250 mm in height.

In one example embodiment, the dissociation well may have a rounded flat bottom or a rounded angled bottom. In an example embodiment, the dissociation wells may have rounded bottoms disposed at an angle of between about 10 degrees and about 45 degrees from horizontal. In an example embodiment, the dissociation wells may have rounded bottoms disposed at an angle of between about 15 degrees and about 30 degrees from horizontal. In exemplary embodiments, the angle may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 degrees. In alternate examples, the tips of the pipettes are angled instead of, or in addition to, the bottom of the wells. The angle of the tip of the pipette may be between about 10 degrees and about 45 degrees. In one example embodiment, the angle of the bottom of the pipette tip is about 45 degrees. In another example, the bottom of the wells and the pipette tips are both angled, such as both angled about 20 degrees.

The wells may be spatially arrayed in a well plate. The well plate may support any suitable number of wells. In certain examples, 6, 12, 24, 48, 96, 384 or 1536 wells are arranged in the well plate. The wells may be arrayed in any suitable arrangement that is compatible with the array of pipettes. The wells may be arrayed under the pipettes in an array of rows and columns. For example, a system that employs 96 wells may have the wells arranged in 8 rows of 12 wells each. In another example, a system that employs 24 wells may have the wells arranged in 4 rows of 6 wells. The well plate may be constructed of any suitable material, such as polypropylene, polystyrene, aluminum, or stainless steel.

The device may include serrated, scalloped, or roughened pipette tips; serrated, scalloped, bubbled, pitted, or roughened bottom surfaces of the wells; or both. The serrations or scallops on the pipette tips or the wells assist with breaking down the tissue to help isolate single cells or single nuclei. In certain examples, the serrations or scallops are cut into the surface of the pipette tip or into the bottom surface of the well to create a jagged, rough, or uneven surface. The serrations or scallops may be comprised of grooves or other indentions to create an uneven surface. In embodiments, the roughened surface may include one or more ridges, grooves, scallops, bumps, or pits. In exemplary embodiments, the one or more bumps or pits may be substantially evenly spaced across the roughened surface internal to each of the plurality of wells. In some examples, the one or more ridges, grooves, or scallops may substantially span a horizontal diameter of each of the plurality of wells. For example, the one or more ridges, grooves, or scallops may be arrayed in a corresponding number of horizontal rows which may be substantially equally spaced apart from a lower end to an upper end of the angled bottom surface. For example, the rows of scalloped ridges may be formed into an angled bottom surface of the well so as to create a “washboard” effect on the angled surface from the lower angled portion to the upper angled portion. It is contemplated within the scope of the disclosure that the one or more ridges, grooves, or scallops may each include one or more channels that form breaks in each of the corresponding number of horizontal rows. In exemplary embodiments, the rows of scalloped ridges may have one or more small channels cut into each row of scalloped ridges, and in some embodiments these one or more small channels may be offset from one another in adjacent rows of scalloped ridges. In other examples, a rounded material is adhered to the bottom surface of the well and/or the surface of the pipette tip, such as glass beads or other material. When the pipette is raised, lowered, and/or twisted, as described herein, the tissue sample is further broken down. For certain tissue samples, the surface should only include rounded or curved uneven surfaces that do not have sharp edges. Sharp edges may damage certain cells or nuclei, so rounded and/or smooth glass beads or other rounded materials may be used to make the surface uneven but not jagged or sharp.

The pipettes may be used to transport a measured volume of fluids, such as the dissociation fluid, to the wells and to remove fluids from the well. The pipettes operate by creating a partial vacuum above the well and selectively releasing the vacuum to draw up or dispense fluids. The vacuum may be created by a pump, by releasing a crimping force on the pipette, or by any other suitable method. The pipettes may be constructed of any suitable laboratory-grade materials, such as glass, polyethylene terephthalate (“PET”), or any other suitable type of plastic or elastomer. The pipette tips of the pipettes are slightly smaller than the diameter of the wells. For example, the wells may have diameters of about 8 mm to about 20 mm, while the tips may have a diameter of about 7 mm to about 19 mm. In other examples, different sizes of the pipette tips may be used, as long as the tips are smaller than the associated wells. For example, a well may be about 8 mm in diameter and the pipette tip that is inserted into the well may be about 7 mm.

A filter may be installed between the pipette and the well. The filter pores may be of any suitable size, such as about 10 micrometers to about 100 micrometers. For the isolation of single cells, filter pore size will be between 60-80 micrometers. For example, for the isolation of single cells, the filter pore size may be about 60, 61, 62, 63, 64, 65, 66, 67, 68, 69, 70, 71, 72, 73, 74, 75, 76, 77, 78, 79, or 80 micrometers. For the isolation of single nuclei, filter pore size will be about 30-40 micrometers. For example, for the isolation of single nuclei, the filter pore size may be about 30, 31, 32, 33, 34, 35, 36, 37, 38, 39, or 40 micrometers. The dissociation fluids, tissue samples, and other desirable fluids and particles pass from the pipette tip through the filter and into the well. Larger particles, contaminants, or any other materials that are larger than the openings on the filter, such as a 200-micrometer particle, will not pass through the filter. Thereby, the filter keeps the sample and the fluids free from contaminants. The filter may be fitted on the top rim of the well or may be placed deeper into the well. The filter may be constructed of filter cloth, nylon mesh, PTFE, or any other suitable material.

Dissociation buffers, or isolation buffers, may be made up of any type of dissociation buffer such as, for example, Buffer 1, Buffer 2, Buffer 3, or combinations thereof. Buffer 1 may include Na₂SO₄ (5.83 g), K₂SO₄ (2.615 g), Glucose (0.905 g), HEPES (1.2 g), 1M MgCl₁₂ (2.5 mL) and ddH₂O added to 500 mL. Buffer 2 may include Buffer 1 (15 mL), 1% Kollidon (0.150 g), 1% TX-100 (150 μl), and 10% BSA (15 μl). Buffer 3 may include Nuclei EZ lysis buffer (Sigma N3408-200ML). Buffer 1, Buffer 2, and/or Buffer 3 may include RNase Inhibitor (non-specific) in tissue dissociation applications where RNase activity is desirable. The buffers may further contain detergents or other agents that serve to break down the cell structure to allow the tissue sample to be dissociated while preserving the nucleus of the cells. Dissociation buffers may include naturally-occurring enzymes, gentler non-enzymatic alternatives, or may work by chelating calcium to prevent cadherins from attaching, releasing cells from surfaces and one another. Cell dissociation reagents may be specific for extracellular matrix substrates.

Dissociation buffers are discussed in greater detail in the following incorporated applications. This application incorporates by reference U.S. Patent Application Publication 2020/0347449 published Nov. 5, 2020, and entitled “Methods for Determining Spatial and Temporal Gene Expression Dynamics During Adult Neurogenesis in Single Cells.” This application incorporates by reference U.S. Provisional Application No. 62/841,408, filed May 1, 2019 and U.S. Provisional Application No. 62/865,829, filed Jun. 24, 2019, both entitled “Methods for Determining Spatial and Temporal Gene Expression Dynamics During Adult Neurogenesis in Single Cells.” This application incorporates by reference PCT Application No. PCT/US2019/055894 filed Oct. 11, 2019, and entitled “Method for Extracting Nuclei or Whole Cells from Formalin-Fixed Paraffin-Embedded Tissues.” This application incorporates by reference U.S. Patent Application Publication 2022/0411783 published Dec. 29, 2022, and entitled “Method for Extracting Nuclei or Whole Cells from Formalin-Fixed Paraffin-Embedded Tissues.” This application incorporates by reference U.S. Provisional Application No. 62/745,259, filed Oct. 12, 2018, entitled “Methods for Extracting and Using Nuclei and Cells from Formalin-Fixed Paraffin-Embedded (FFPE) Tissue”; U.S. Provisional Application No. 62/813,634, filed Mar. 4, 2019, entitled “Methods for Extracting and Using Nuclei and Cells from Formalin-Fixed Paraffin-Embedded (FFPE) Tissue and Use of Biomarkers Identified”; U.S. Provisional Application No. 62/829,402, filed Apr. 4, 2019, entitled “Methods for Extracting and Using Nuclei and Cells from Formalin-Fixed Paraffin-Embedded (FFPE) Tissue and Use of Biomarkers Identified”; U.S. Provisional Application No. 62/887,339, filed Aug. 15, 2019, entitled “Methods for Extracting and Using Nuclei and Cells from Formalin-Fixed Paraffin-Embedded (FFPE) Tissue and Use of Biomarkers Identified”; and U.S. Provisional Application No. 62/890,971, filed Aug. 23, 2019, entitled “Methods for Extracting and Using Nuclei and Cells from Formalin-Fixed Paraffin-Embedded (FFPE) Tissue and Use of Biomarkers Identified”. The entire contents of the above-identified applications are hereby fully incorporated herein by reference.

### Example Embodiments and Methods of Use

The dissociation wells, modified pipette tips, and kits comprising the same may be used with manual pipettors, or with robotic liquid handling systems, which may include temperature regulating devices and/or systems to control temperature during the process of isolating single cells and/or nuclei (e.g., by cooling fluids, pipette tips, tissue dissociation well plates, etc. that are used in liquid handling systems described herein). The following section provides an overview of how the dissociation wells may be used, example protocols, and an example apparatus for carrying out said protocols using the dissociation wells and modified pipette tips.

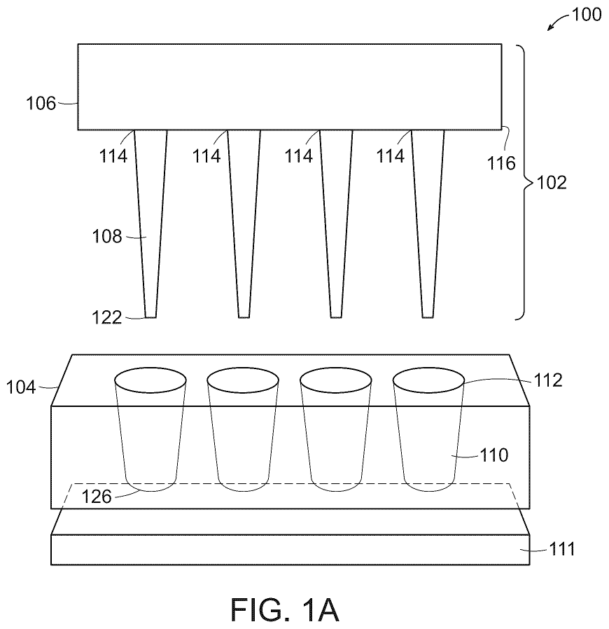

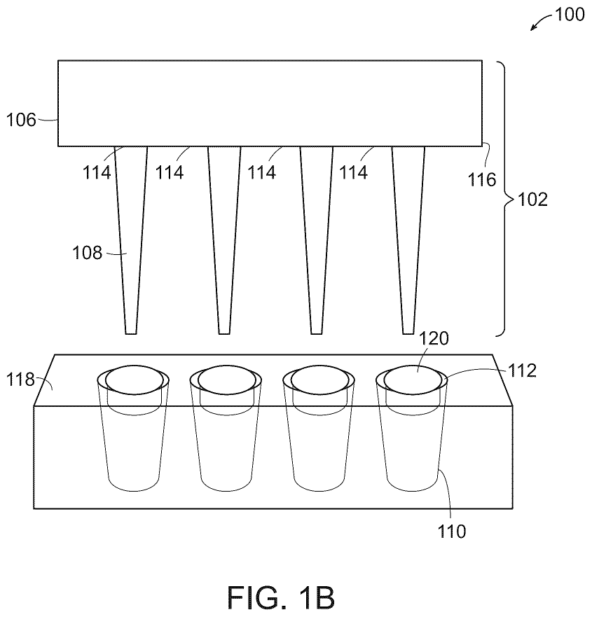

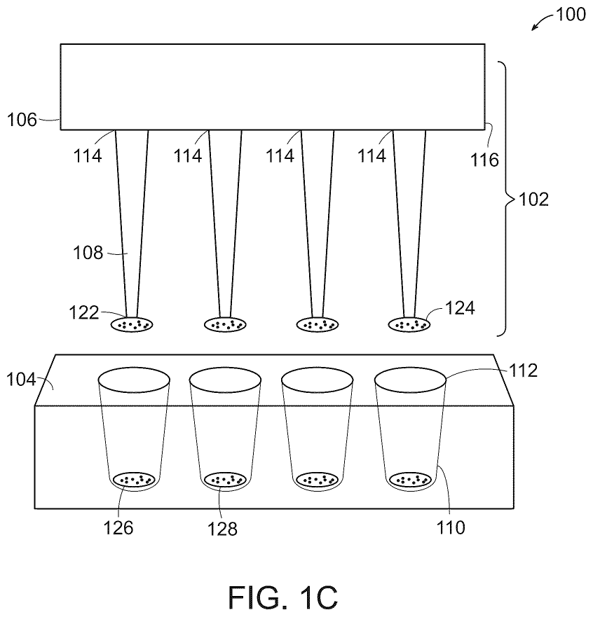

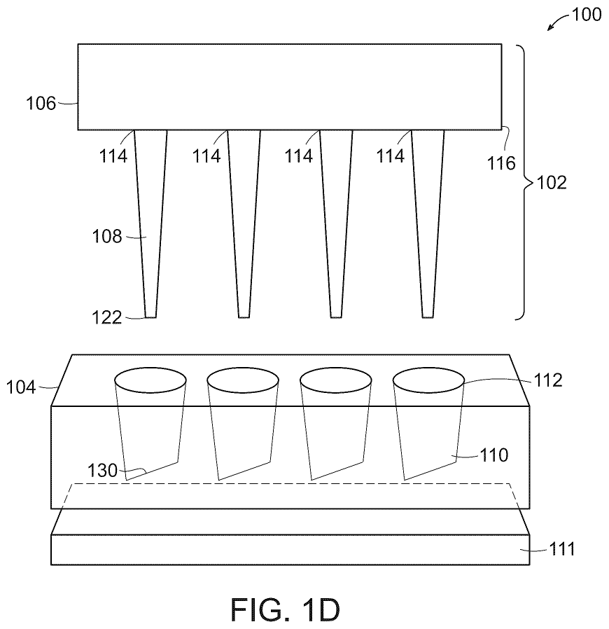

FIG. 1A illustrates an exemplary embodiment of single-cell (or single nuclei) isolation pipettor system 100 including distal pipettor end 102 and dissociation well plate 104. Distal pipettor end 102 includes pipette tip adaptor 106 and pipette tip (or tips) 108 each having a distal pipette end 122. Dissociation well plate 104 includes one or more wells 110, each of which includes a well opening 112 and a lower well end 126. FIG. 1A illustrates an exemplary isolation pipettor system 100 including a set of four pipette tips 108 delivering dissociation fluids to a set of four wells 110 via four well openings 112, where each well opening 112 reversibly interacts with a specific pipette tip 108. The pipette tips 108 are permanently or reversibly mated to pipette tip adaptor 106. In embodiments, pipette tips 108 may be reversibly mated to pipette adaptor 106 via one or more mounting areas 114 fixed to distal adaptor end 116. Mounting areas 114 may be positioned to allow a desired number and configuration of pipette tips 108 to be removably affixed to pipette tip adaptor 106. The pipette tip adaptor 106 may lower the pipette tips 108 into the wells 110 and also raise the pipette tips 108 out of wells 110 in a reversible manner. Mounting areas 114 are configured to receive different pipette tips 108, such as by a threaded or locking engagement. In some exemplary embodiments, the isolation pipettor system 100 may include temperature regulation plate 111, which may function to heat or cool dissociation well plate 104. For example, temperature regulation plate 111 may cool well plate 104 (e.g., to about −10° C. to about 10° C.) to enhance the process of isolating single cells or single nuclei. In some exemplary embodiments, the temperature regulation plate 111 may cool well plate 104 to about 4° C.

FIG. 1B illustrates an exemplary embodiment of single-cell (or single nuclei) isolation pipettor system 100 in which distal pipettor end 102 is positioned over final well plate 118, which includes a filter 120 seated in well opening 112 of well 110.

FIG. 1C illustrates an exemplary embodiment of single-cell (or single nuclei) isolation pipettor system 100 in which distal pipette ends 122 of the pipette tips 108 are modified to include roughened tip surfaces 124, which may include abrasive materials, serrations, rippled edges, and the like to facilitate the process of breaking down a sample (e.g., a tissue, specimen, etc.) to isolate single cells or single nuclei. In exemplary embodiments, roughened tip surfaces 124 include serrations cut into the distal pipette ends 122 of the pipette tips 108 to create a jagged, rough, or uneven surface. In other examples, an abrasive material (e.g., plastic beads, glass beads, abrasive particles, and the like) may be adhered to the distal pipette ends 122 of the pipette tips 108 to create roughened tip surfaces 124.

In exemplary embodiments, dissociation well plate 104 may include wells 110 in which lower well end 126 includes roughened well surfaces 128, which may include abrasive materials, serrations, wave-like surfaces, rippled edges, and the like, as shown in FIG. 1C. For example, roughened well surfaces 128 may include serrations created with grooves cut or etched into lower well end 126 to create an uneven surface. In other examples, an uneven or abrasive material may be adhered to the upper surface of the lower well end 126 (e.g., plastic beads, glass beads, abrasive particles, and the like). It is contemplated within the scope of the disclosure that single-cell (or single nuclei) isolation pipettor system 100 may include: 1) roughened tip surfaces 124 on pipette tips 108, 2) wells 110 in which lower well end 126 includes roughened well surfaces 128, 3) both roughened tip surfaces 124 on pipette tips 108 and wells 110 in which lower well end 126 includes roughened well surfaces 128, or 4) neither roughened tip surfaces 124 on pipette tips 108 nor wells 110 in which lower well end 126 includes roughened well surfaces 128. One of skill in the art will appreciate that certain tissue samples will require that roughened tip surfaces 124 and/or roughened well surfaces 128 may require rounded, uneven surfaces that do not include sharp edges that may damage certain types of cells or nuclei. For dissociation of single cells or nuclei from such more delicate tissue types, it is contemplated that rounded glass beads or other rounded materials or rounded edges may be used to make the surface uneven without being jagged or sharp.

As described in further detail herein, pipette tip or tips 108 may be raised, lowered, and/or twisted relative to wells 110 on dissociation well plate 104 to facilitate breakdown of a sample (e.g., a tissue sample) to generate single cells and/or single nuclei.

The wells 110 may be arrayed in the dissociation well plate 104 such that when each of the pipette tips 108 are lowered, the respective distal pipette ends 122 of the pipette tips 108 are touching substantially the center portion of the inner bottom surface of lower well end 126 of the wells 110. The dissociation well plate 104 may support any suitable number of wells 110. For example, 6, 12, 24, 48, 96, 384 or 1536 wells 110 may be arranged in the dissociation well plate 104. In certain examples, the wells 110 may have a volume capacity of about 2 mL to about 10 mL and a height of about 20 mm to about 60 mm. In other exemplary embodiments, wells 110 may have different sizes that vary based on the size of the pipette tip 108 and/or the size of the tissue sample. For example, in applications in which excess froth is generated in the dissociation and single cell/nuclei isolation process, a well 110 may have a larger volume capacity that may be used to keep the froth from spilling out of the wells and contaminating other samples. In exemplary embodiments, well 110 may be approximately 250 mm in diameter and approximately 250 mm in height.

The distal pipette ends 122 and/or roughened tip surfaces 124 of the pipette tips 108 may be slightly smaller than the diameter of the wells 110. For example, the wells 110 may have diameters of about 8 mm to about 20 mm while the distal pipette ends 122 and/or roughened tip surfaces 124 may have a diameter of about 7 mm to about 19 mm. In other exemplary embodiments, different sizes of the distal pipette ends 122 and/or roughened tip surfaces 124 may be used, as long as the distal pipette ends 122 and/or roughened tip surfaces 124 are smaller than the associated wells 110. For example, a well 110 may be about 8 mm in diameter and the distal pipette ends 122 and/or roughened tip surfaces 124 that are inserted into the well 110 may be about 7 mm.

As shown in FIG. 1B, each of wells 110 may be fitted with a filter 120 seated in well opening 112 of each well 110. In exemplary embodiments, filters 120 may be seated at any of a variety of positions that are lower than the top of well opening 112, e.g., closer to lower well end 126.

Filters 120 may include a plurality of filter pores of any suitable size, such as about 10 micrometers to about 100 micrometers. The dissociation fluids, tissue samples, and other desirable fluids and particles pass from the distal pipette ends 122 and/or roughened tip surfaces 124 through the filter 120 and into the wells 110. Larger particles, contaminants, or any other materials that are larger than the openings on filter 120, such as, e.g., a 200-micrometer particle, will not pass through the filter 120, which thereby keeps the sample and the fluids free from contaminants.

FIG. 1D shows a cross-section view of an exemplary isolation pipettor system including a temperature regulation plate and wells with angled bottom surfaces 130. As shown in FIG. 1D, angled bottom surfaces 130 may have a lower side (shown on the left side of each well) and an upper side (shown on the right side of each well) so that a portion of the circumferential wall adjoining the upper side of the angled bottom surface is shorter than a portion of the circumferential wall adjoining the lower side of the angled bottom surface.

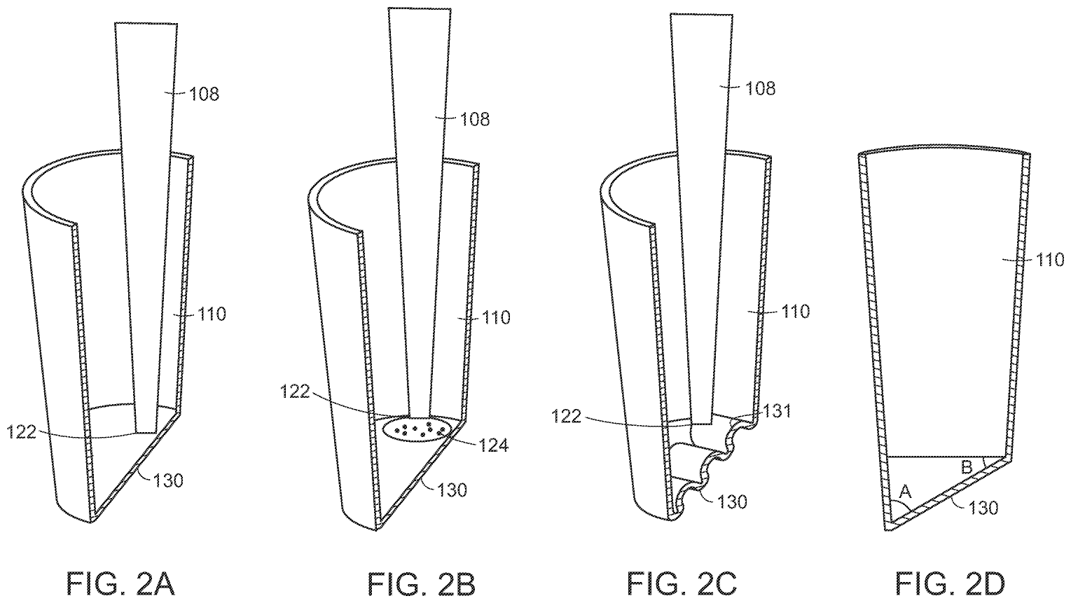

FIG. 2A illustrates an exemplary embodiment in which a pipette tip 108 is delivering dissociation fluids to a well 110 with an angled bottom surface 130. As illustrated, the pipette tip 108 is lowered into the well 110 to allow the distal pipette end 122 of the pipette tip 108 to reach the angled bottom surface 130 of the well 110. In exemplary embodiments, the angled bottom surface 130 may be angled such that the distal pipette end 122 does not lie flat against the angled bottom surface 130 when lowered.

FIG. 2B illustrates an exemplary embodiment in which a pipette tip 108 having roughened tip surface 124 at distal pipette end 122 is delivering dissociation fluids to a well 110 with an angled bottom surface 130. As illustrated, the pipette tip 108 is lowered into the well 110 to allow the distal pipette end 122 and roughened tip surface 124 of the pipette tip 108 to reach the angled bottom surface 130 of the well 110.

The difference in the angle of the distal pipette ends 122 and/or roughened tip surfaces 124 and the angled bottom surface 130 allows the tissue to be broken down when the distal pipette ends 122 and/or roughened tip surfaces 124 shown in FIGS. 2A-2B are twisted.

FIG. 2C illustrates an exemplary embodiment in which a pipette tip 108 is delivering dissociation fluids to a well 110 with an angled bottom surface 130 having roughened well surface 131. In the exemplary embodiment shown in FIG. 2C, roughened well surface 131 is scalloped, however, in other embodiments roughened well surface 131 may be ridged, bubbled, pitted, and the like.

FIG. 2D illustrates a front, cross-sectional view of well 110 shown in FIGS. 2A-2B and illustrates that angled bottom surface 130 is associated with angle A and angle B, which may be any suitable angle that allows the distal pipette end 122 (with or without roughened tip surface 124) to break down a sample (e.g., tissue sample) when twisted. In an exemplary embodiment, the angle A or angle B of the angled bottom surface 130 may be between about 10 degrees and about 40 degrees. In exemplary embodiments, angle A or angle B of angled bottom surface 130 may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 degrees, or any decimal value between any of the aforementioned integer degree values.

In exemplary embodiments in which the angle of the distal pipette ends 122 and/or roughened tip surfaces 124 is different than 90 degrees from a vertical axis of the pipette tip 108 (e.g., the axis from a proximal end to a distal end of pipette tip 108), the angle of the distal pipette ends 122 and/or roughened tip surfaces 124 may be any suitable angle that allows the distal pipette end 122 (with or without roughened tip surface 124) to break down a sample (e.g., tissue sample) when twisted. In an exemplary embodiment, the angle of the distal pipette ends 122 and/or roughened tip surfaces 124 may be between about 10 degrees and about 40 degrees. In exemplary embodiments, the angle of distal pipette ends 122 and/or roughened tip surfaces 124 may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 degrees, or any decimal value between any of the aforementioned integer degree values.

In an exemplary embodiment, the well 110 is constructed with a height of about 20 mm to about 60 mm and a diameter of about 8 mm to about 20 mm. Other heights and diameters may be used for certain types of tissue samples or dissociation processes. In other exemplary embodiments, the distal pipette ends 122 and/or roughened tip surfaces 124 are angled instead of the angled bottom surface 130 of the well 110. In other words, lower well end 126 of well 110 is internally rounded as shown in FIG. 1A.

Different types of tissue samples may require different angles for the angled bottom surface 130. Similarly, different types of tissue samples may require different angles for the distal pipette ends 122 and/or roughened tip surfaces 124 in embodiments in which the angle of the distal pipette ends 122 and/or roughened tip surfaces 124 is different than 90 degrees from a vertical axis of the pipette tip 108 as described above. For example, when using certain frozen tissue samples, the wells 110 may have angled bottom surfaces 130 between about 10 degrees and about 40 degrees (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 degrees), while for certain fresh samples rounded bottom surfaces 130 are preferred. The elasticity, toughness, and texture of the tissue sample may dictate the angle of the angled bottom surface 130 and/or the angle of distal pipette ends 122 and/or roughened tip surfaces 124.

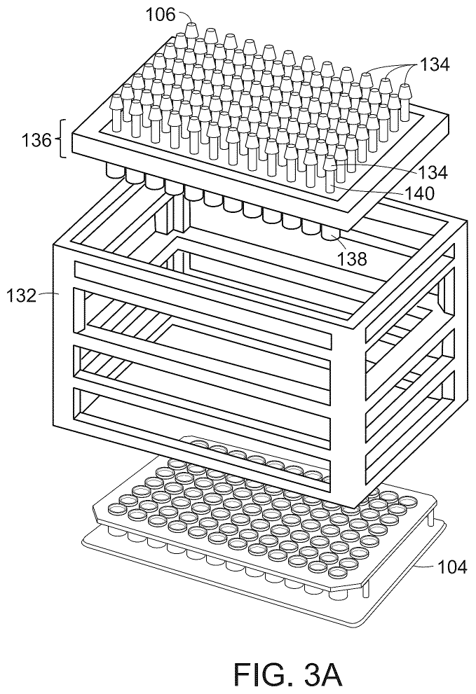

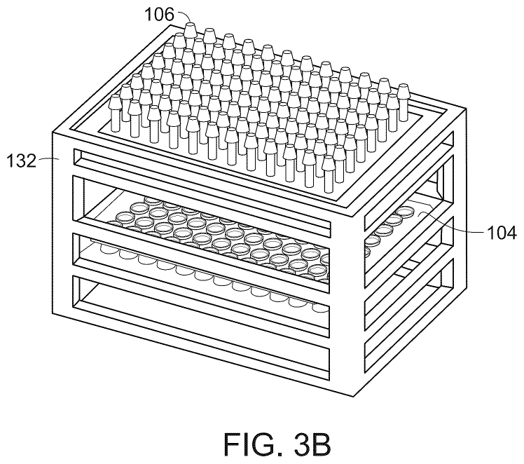

FIGS. 3A-3B illustrate an exemplary embodiment in which pipette tip adaptor 106 is reversibly coupled to dissociation well plate 104 via adaptor frame 132.

FIG. 3A provides an exploded view of pipette tip adaptor 106, dissociation well plate 104, and adaptor frame 132. In an exemplary embodiment, pipette tip adaptor 106 may be configured to accommodate 96 pipette tips 108, which would interface with a dissociation well plate 104 configured with 96 wells 110. Pipette tip adaptor 106 may include a plurality of through bore holes 134 that traverse the short thickness 136 of pipette tip adaptor 106. Each bore hole 134 is associated with a tip interface 138 configured to reversibly receive a pipette tip 108 and a pipettor interface 140 configured to interface with an individual channel (e.g., an air channel or a pressure channel) in a manual or a robotic pipettor (not shown). The pipette tips 108 may each be reversibly inserted onto tip interface 138. The tip interface 138 supports and secures the pipette tip 108 to pipette tip adaptor 106 to allow the pipette tip(s) 108 to be moved particular distances relative to dissociation well plate 104. For example, the pipettes may be moved vertically downward to place each pipette tip 108 in contact with the lower well end 126 or angled bottom surface 130 of a well 110, as well as moved vertically upward out of the well 110.

The pipette tip adaptor 106 may further provide a force to twist the pipette tips 108. Each pipette tip 108 may be individually twisted by the pipette tip adaptor 106 to provide a force to break down the tissue sample. In exemplary embodiments, the pipette tips 108 may be twisted between about 90 degrees and about 360 degrees. In an example, each time the pipette tip 108 is lowered into the well 110, the pipette tip 108 is twisted about 180 degrees. The twisting action may be created by a bi-directional motor on the pipette tip adaptor 106 to independently twist each pipette tip 108. In another example, a single motor may drive linkages to twist each of the pipette tips 108 in the adaptor 106. The pipette tips 108 may be configured to twist simultaneously or independently of each other.

The pipette tip adaptor 106 may be placed into an adaptor frame 132 that supports the pipette tip adaptor 106 at a desired height such that when the pipette tip 108 is lowered into the well 110, the distal pipette ends 122 and/or roughened tip surfaces 124 of the pipette tip 108 reach near the lower well end 126 or the angled bottom surface 130 of well 110. The adaptor frame 132 causes the pipette tip adaptor 106 to be positioned such that when lowered, the pipette tip adaptor 106 places each pipette tip 108 in contact with the lower well end 126 or the angled bottom surface 130 of a well 110. The adaptor frame 132 may be slidably placed over the well plate 104 to ensure proper positioning in each direction.

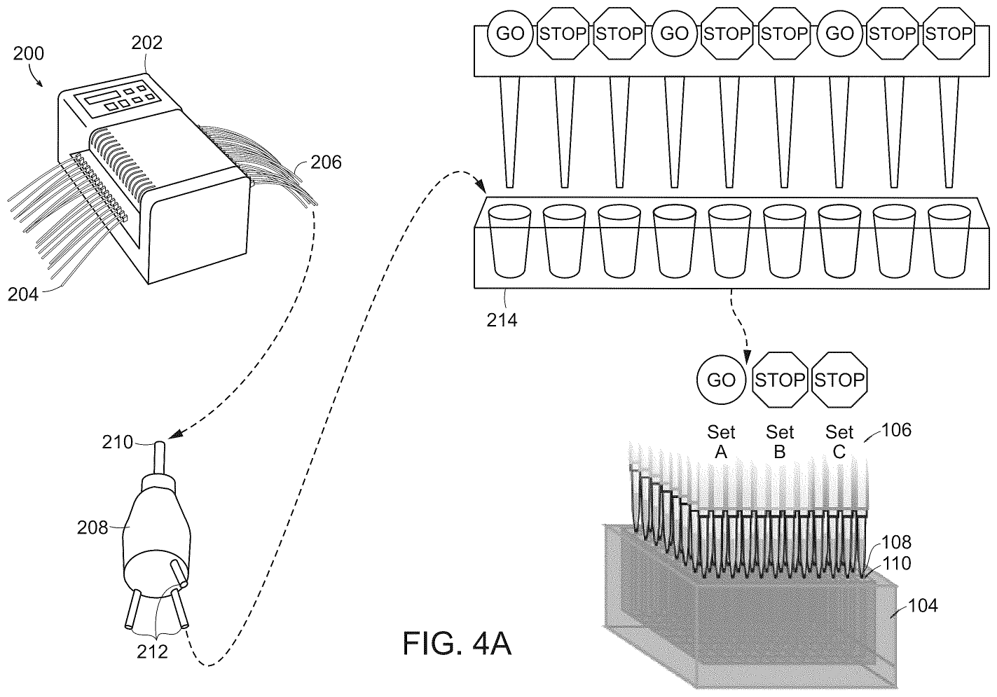

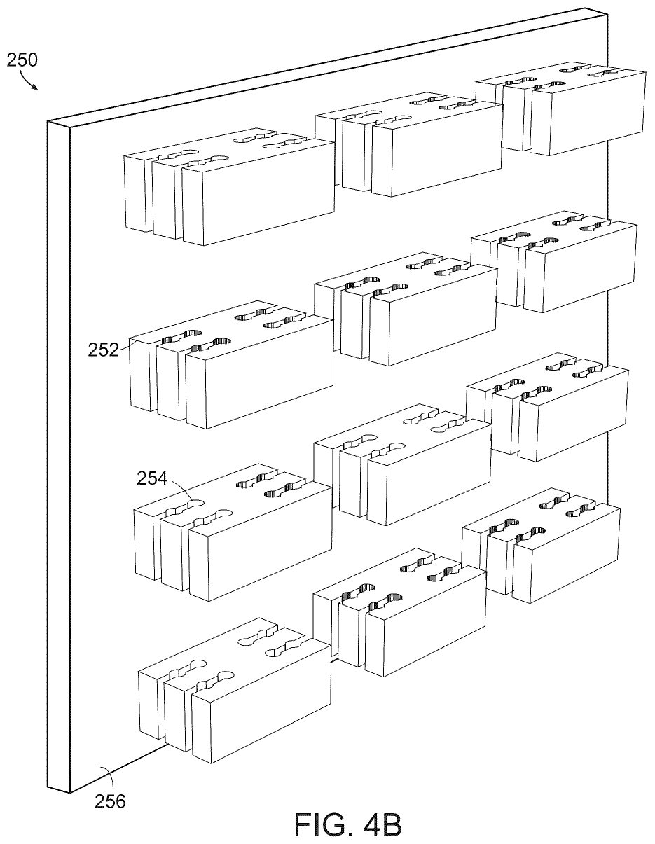

FIG. 4A is an illustration of a dissociation device 200 with a pump 202, 3-way perfusion manifold 208, and valve controllers 214. Pump 202 includes pump inlets 204 and pump outlets 206. 3-way perfusion manifold 208 includes manifold inlet 210 and two or more (e.g., 3) manifold outlets 212. The dissociation device 200 may include some or all of the components listed herein. In certain exemplary embodiments, the dissociation device 200 may omit the manifold 208 and provide one or more additional pumps and/or one or more additional pump outlets 206 with associated tubing to deliver the fluids directly to the valve controllers 214.

The pump 202 may be a peristaltic pump, a roller pump, or any other type of positive displacement pump. The pump 202 receives one or more fluids in one or more pump inlets 204 to the pump 202 and provides a positive force to pump the fluid from the pump inlets 204, through the pump 202, and out through pump outlets 206. The pump 202 may alternatively, or additionally, provide a suction force to the pipette tips 108 (ultimately in fluid connection with pump outlet 206) by reversing the flow of the fluids. That is, the pump 202 may create a suction on the pipette tip 108 side of the pump 202 and provide a positive flow to the side of the pump 202 including inlet 204. By pumping in the opposite direction, the pump 202 forces the pipette tips 108 to suction materials out of the wells 110 positioned in dissociation well plate 104. As disclosed herein, dissociation device 200 may also be used to add/transfer fluids into final well plate 118 (e.g., by moving processed fluids from dissociation well plate 104 to final well plate 118).

In exemplary embodiments, the pump 202 provides 16 pump outlets 206 (with associated tubing). In exemplary embodiments, pump 202 may be configured with a greater or lower number of pump inlets 204 and/or pump outlets 206. In examples, more than one pump 202 may be used to achieve the desired number of pump inlets 204 and/or pump outlets 206. The number of pump outlets 206 is configured based on the number of wells 110 in dissociation well plate 104 and pipette tips 108 that need to be supplied.

In an exemplary embodiment, 96 wells 110 are being used to dissociate 96 tissue samples (see e.g., FIG. 4A, bottom right). Accordingly, 96 pipette tips 108 attached to pipette tip adaptor 106 may be used, and pump 202 may ultimately control fluid flow to 96 fluid tubes which are in fluid connection with pipette tips 108. The pump 202 may deliver the fluids to a 3-way perfusion manifold 208 via the pump outlets 206 either directly by connection to manifold inlet 210 or indirectly via tubing connecting pump outlets 206 to manifold inlets 210. The tubes associated with pump outlets 206 provide the fluids to a manifold inlet 210 of the 3-way perfusion manifold 208. The 3-way perfusion manifold 208 splits the flow of the fluid into three manifold outlets 212. The manifold outlets 212 deliver the fluids to the valve controller 214 via the positive flow pressure from the pump 202. In alternate embodiments, any other type of flow splitting device may be used. In other exemplary embodiments, the number of manifold outlets 212 may be greater than (e.g., 4, 5, 6, 7, 8, etc.) or less than (e.g., 2) three.

In an example, the 3-way perfusion manifold 208 allows a pump 202 with only 16 pump outlets 206 to provide fluids to 48 valve controllers 214. Any suitable number of manifold outlets 212 or 3-way perfusion manifolds 208 may be used to provide the required fluids to valve controllers 214. As disclosed herein, in one embodiment, two pumps 202 with 16 pump outlets 206 each may be provided to 32 3-way perfusion manifolds 208, which may then split the 32 pump outlets 206 into 96 tubes for delivery to the valve controllers 214.

The manifold outlets 212 of the 3-way perfusion manifold 208 may deliver the fluids to the valve controllers 214, which then control the flow of the fluid to each individual pipette tip 108.

The fluids may flow to the pipette tips 108 at a time when more dissociation fluid should be injected into the wells 110 or at a time when it is desirable to expel extracted tissue samples out of the pipette tips 108.

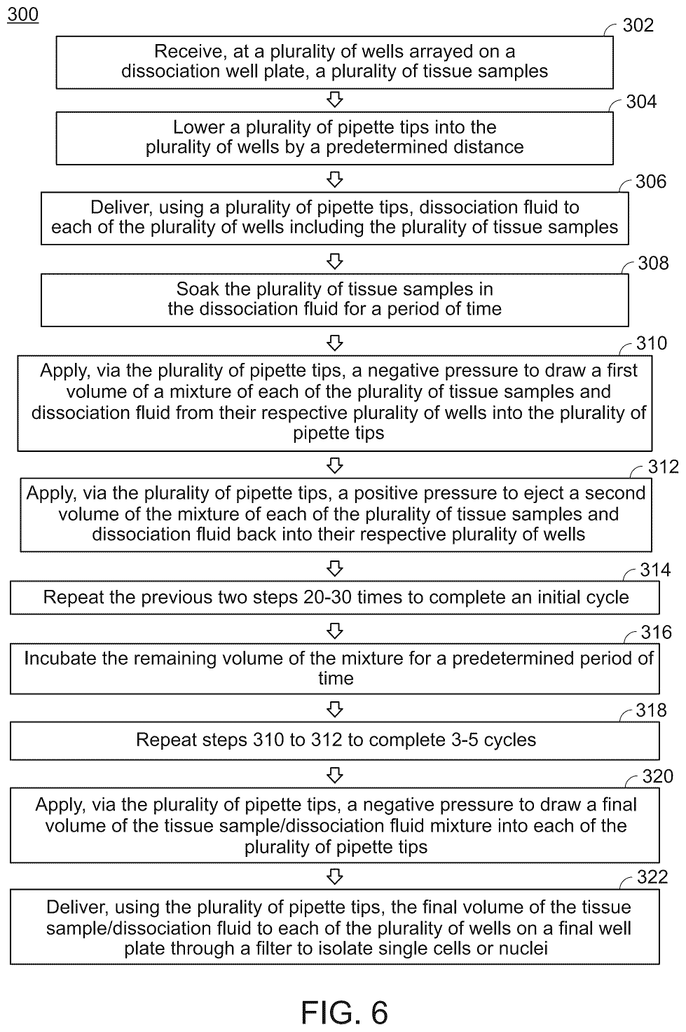

As shown in FIG. 4B, the valve controllers 214 may control or instruct pinch valve system 250, which includes pinch valves 252 having pinch orifices 254 mounted on pinch valve plate 256 (see e.g., FIG. 6, described below) to open or close to allow fluid flow, or prevent fluid flow, respectively, to the pipette tips 108.

The valve controllers 214 may be any type of valve controller such as a pneumatic actuator or solenoid-based pinch valves. Any other suitable type of valve may be used. The valve controllers 214 may receive instructions on when to operate, such as by a central control device (not shown), such as a computing device or a stand-alone device control device, known to one of skill in the art. Pinch valve 252 include adjustable pinch orifices 254. For example, pinch orifices 254 may range from fully closed (0% open) to fully open (100% open), or any percentage of partial opening therebetween (e.g., 10%, 20%, 30%, 40%, 50%, 60%, 70%, 80%, 90%, etc., or any intermediate percentage values therebetween, e.g., 1%, 2%, 3%, 4%, 5%, 6%, 7%, 8%, 9%, or 11%, 12%, 13%, 14%, 15%, 16%, 17%, 18%, 19%, and the like). The valve controllers 214 may cause the pinch valves 252 to open fully when instructed or the valve controllers 214 may throttle the pinch valves 252 to open to only a percentage of a fully open position. In the continuing example, 96 valve controllers 214 open 96 valves to allow the fluid to flow to 96 pipette tips 108. Valve controllers 214 may open the pinch valves 252 in a controlled manner, such as simultaneously or sequentially.

The valve controllers 214 may open the pinch valves 252 to allow fluid to flow into the pipette tips 108 from the pump 202 or the pinch valves 252 may open to allow a suction force from the pump 202 to extract material from the wells 110. The valve controllers 214 may utilize any control mechanisms or signals to control the pinch valves 252. For example, a computing device, manual switches, or any type of device controls may provide instructions or signals to the valve controllers 214 to enable pinch valves 252 to open to any of a plurality of positions between 0% and 100% open.

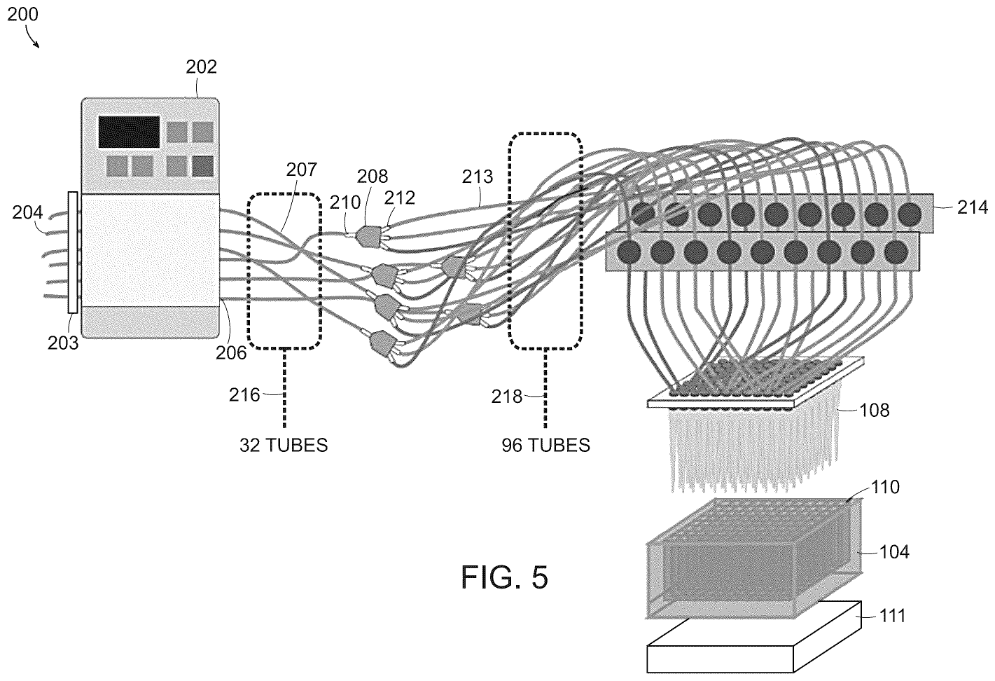

In one example shown in FIG. 5, the manifold outlet tubes 213 provide a fluid connection from the 3-way perfusion manifolds 208 to the pipette tips 108 while passing through the pinch valve system 250. When the pinch valves 252 are actuated by the valve controller 214, the pinch valves 252 may close and stop flow of the dissociation fluid to or from the pipette tips 108. In one example shown in FIG. 4B, each pinch valve 252 controls four tubes through each of four pinch orifices 254. The pinch valves 252 may be controlled by the valve controllers 214. In some exemplary embodiments, the dissociation device 200 may include temperature regulation plate 111 and/or temperature regulation module 203, both of which may function to heat or cool aspects of the device and system. For example, temperature regulation plate 111 may regulate the temperature of dissociation well plate 104 by maintaining a cooling temperature of about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., or 10° C. Similarly, temperature regulation module 203 may regulate the temperature of fluids (e.g., dissociation fluids) moving through pump inlet 204 and/or pump outlet 206 and associated outlet tubes 207 by maintaining a cooling temperature of about −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., or 10° C. While temperature regulation module 203 is shown in the exemplary embodiment of FIG. 5 as being positioned on the pump inlet 204 side of pump 202 so that the plurality of pump inlets 204 pass through temperature regulation module 203, it is contemplated within the scope of the disclosure that temperature regulation module 203 may also be positioned proximate to pump outlets 206, or anywhere between pump outlets 206 and pipette tips 108. Temperature regulation plate 111 and/or temperature regulation module 203 may incorporate any of a variety of temperature regulation systems such as, for example, a Peltier system.

Referring back to FIG. 4A, pipette tips 108 may be positioned over a well plate 104 with 96 wells 110. The tubes illustrated as being fed into the pipette tips 108 are divided into three sets: Set A, Set B, and Set C. In the illustrative example, only one of the three sets is being fed fluids at a time. The other two sets are either holding fluid or allowing the tissue sample to soak in the dissociation fluid. The number of different fluids being pumped may be controlled by the tissue sample(s) that is being dissociated. The fluids may be made up of any type of dissociation buffer. The fluid may further contain detergents or other agents that serve to break down the cell structure to allow the tissue sample to be dissociated while preserving the nucleus of the cells.

FIG. 5 is an illustration of an exemplary dissociation device 200 including 3-way perfusion manifolds 208 configured to deliver fluid to 96 pipette tips 108. The dissociation device 200 operates substantially as described in FIGS. 4A-4B. FIG. 5 depicts a set of outlet tubes 207 that provide fluid connections between pump outlets 206 and 3-way perfusion manifold 208 via manifold inlet 210. 3-way perfusion manifold 208 divides the fluid flow into three fluid paths that exit 3-way perfusion manifold 208 via manifold outlets 212, which provide fluid connections to pipette tips 108 via manifold outlet tubes 213 that pass through the valve controllers 214 and pinch valves 252. In the illustrative embodiment shown in FIG. 5, six output tubes 207 exit pump 202 and connect to six 3-way perfusion manifolds 208 via six manifold inlets 210. After being split in the 3-way perfusion manifold 208, the fluid flows are directed into 18 manifold outlet tubes 213 that pass through valve controller 214 as shown in FIG. 5.

As annotated in FIG. 5, if the six outlet tubes 207 were to be expanded to 32 pump outlet tubes 216, then the number of 3-way perfusion manifold 208 would increase to 32, resulting in the 18 manifold outlet tubes being increased to 96 manifold outlet tubes 218, which would then pass through an equivalent number of valve controllers 214.

The 96 tubes, of which only 18 are illustrated in FIG. 5, enter the valve controllers 214 to be pumped through to the pipette tips 108. The pipette tips 108 are illustrated as being reversibly attached to the pipette tip adaptor 106 as described above. The pipette tips 108 are suspended over the well plate 104 containing 96 wells 110.

FIG. 6 presents a block flow diagram depicting a high-throughput parallel processing titration dissociation method 300 for dissociating tissue samples and isolating single cells or single nuclei. Briefly, titration dissociation method 300 involves receiving, at a plurality of wells 110 arrayed on a dissociation well plate 104, a plurality of tissue samples as shown in step 302. A plurality of pipette tips 108 are then lowered into the plurality of wells by a predetermined distance as shown in step 304. The plurality of pipette tips 108 is then used to deliver dissociation fluid to each of the plurality of wells 110, which include the plurality of tissue samples as shown in step 306. The plurality of tissue samples is then soaked in the dissociation fluid for a period of time as shown in step 308. In step 310, the plurality of pipette tips 108 then apply a negative pressure to draw a first volume of a mixture of each of the plurality of tissue samples and dissociation fluid from their respective plurality of wells 110 into the plurality of pipette tips 108. In step 312, the plurality of pipette tips 108 then apply a positive pressure to eject a second volume of the mixture of each of the plurality of tissue samples and dissociation fluid back into their respective plurality of wells 110. Steps 310 and 312 may then be repeated sequentially for about 20-30 times to complete an initial cycle at step 314. In step 316, the remaining volume of the mixture is incubated for a predetermined period of time. At step 318, steps 310 to 312 are then repeated to complete 3-5 cycles including the step 316 incubation step between each cycle. In step 320, the plurality of pipette tips 108 apply a negative pressure to draw a final volume of the tissue sample/dissociation fluid mixture into each of the plurality of pipette tips 108. In step 322, the plurality of pipette tips 108 are used to deliver the final volume of the tissue sample/dissociation fluid to each of a plurality of wells 110 on a final well plate 118 through a filter 120 to isolate single cells or single nuclei.

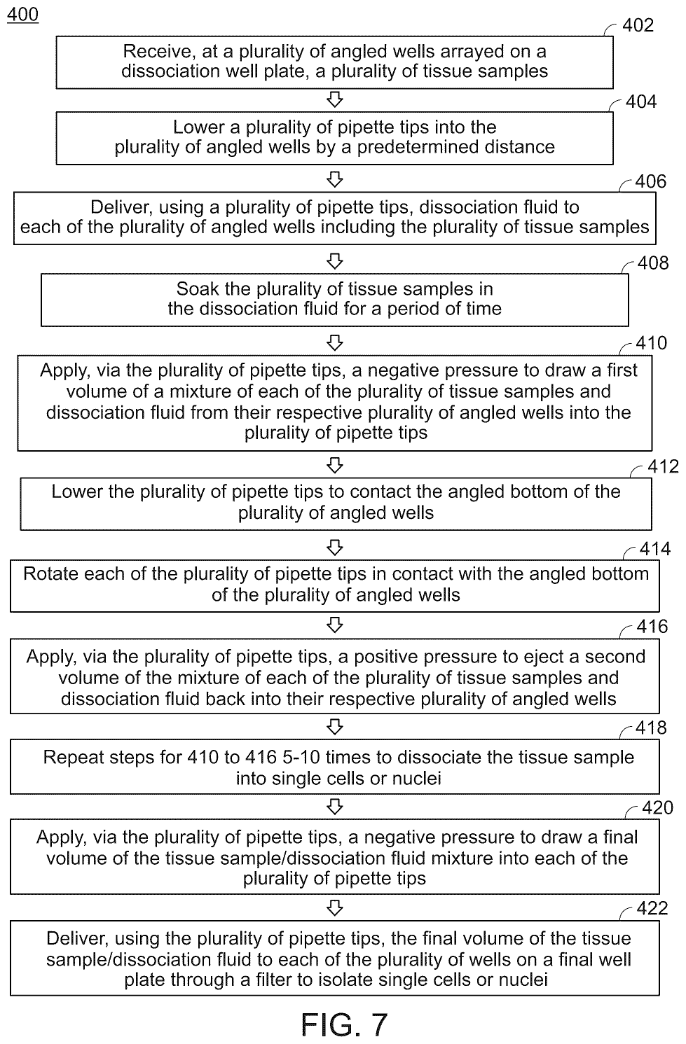

FIG. 7 presents a block flow diagram depicting a high-throughput optimized dissociation method 400 for dissociating tissue samples and isolating single cells or single nuclei using a well plate 104 having wells 110 with angled bottom surfaces 130. Briefly, optimized dissociation method 400 involves receiving, at a plurality of wells 110 with angled bottom surfaces 130 arrayed on a dissociation well plate 104, a plurality of tissue samples as shown in step 402. A plurality of pipette tips 108 are then lowered into the plurality of wells 110 with angled bottom surfaces 130 by a predetermined distance as shown in step 404. The plurality of pipette tips 108 is then used to deliver dissociation fluid to each of the plurality of wells 110 with angled bottom surfaces 130, which include the plurality of tissue samples as shown in step 406. The plurality of tissue samples is then soaked in the dissociation fluid for a period of time as shown in step 408. In step 410, the plurality of pipette tips 108 then apply a negative pressure to draw a first volume of a mixture of each of the plurality of tissue samples and dissociation fluid from their respective plurality of wells 110 with angled bottom surfaces 130 into the plurality of pipette tips 108. In step 412, the plurality of pipette tips 108 are further lowered until the distal pipette ends 122 of the plurality of pipette tips 108 are in contact with the angled bottom surfaces 130 of wells 110. In step 414, each of the plurality of pipette tips 108 are rotated back and forth in a clockwise and counterclockwise direction while in contact with the angled bottom surfaces 130 of wells 110. In step 416, the plurality of pipette tips 108 then apply a positive pressure to eject a second volume of the mixture of each of the plurality of tissue samples and dissociation fluid back into their respective plurality of angled wells 110. In step 418, steps 410 to 416 are repeated 5-10 times to progressively dissociate the tissue sample into isolated single cells and/or single nuclei. In step 420, the plurality of pipette tips 108 apply a negative pressure to draw a final volume of the tissue sample/dissociation fluid mixture into each of the plurality of pipette tips 108. In step 422, the plurality of pipette tips 108 are used to deliver the final volume of the tissue sample/dissociation fluid to each of a plurality of wells 110 on a final well plate 118 through a filter 120 to isolate single cells or single nuclei.

In titration dissociation method 300 and optimized dissociation method 400, a lab technician or other operator delivers tissue samples to a plurality of wells 110 arrayed on a well plate 104. In optimized dissociation method 400, the wells 110 may being optimized for isolating single cells or single nuclei by included angled bottom surfaces 130. The tissue samples may be frozen, fresh tissue samples, or formaldehyde fixed tissue samples. The technician may place the tissue samples on the angled bottom surface 130 of the wells 110.

The dissociation device performing the dissociation of the tissue samples may employ any number of wells 110 and tissue samples. In certain examples, 6, 12, 24, 48, 96, 384 or 1536 wells 110 are arranged in the dissociation well plate 104. By dissociating these numbers of tissue samples concurrently, a single dissociation device may produce a significantly greater number of single-cell or single-nuclei samples for sequencing or for any other purpose than may be produced by conventional practices. In examples described throughout, while the operations of a single pipette tip 108, well 110, or other component may be described, the operations may be performed by any number of components concurrently.

In methods 300 and 400, the dissociation device delivers, via each of the pipette tips 108, dissociation fluid to each of the wells 110. The dissociation fluids may be made up of any type of dissociation buffer. The fluid may further contain detergents or other agents that serve to break down the cell structure to allow the tissue sample to be dissociated while preserving the nucleus of the cells. The dissociation device may provide the fluids based on the components described at least in FIGS. 4A-4B and FIG. 5 as discussed herein.

The dissociation fluid may be injected into the wells 110 containing the tissue samples. The tissue samples may be allowed to soak in the dissociation fluid for a configured amount of time. In examples, the soaking time may be ten seconds, one minute, or ten minutes, depending on the type of tissue, the type of dissociation fluid, or other factors.

In method 300 and/or 400, at step 310 or 410, the dissociation device withdraws, via each of the pipette tips 108, tissue samples and some or all of the dissociation fluid from each of the wells 110 with a negative pressure (e.g., suction) on each of the pipette tips 108. The suction may be provided to the pipette tips 108 by any suitable action by pump 202 or in combination with the valve controller 214 and pinch valve system 250 discussed herein. In an illustrative example, the pipette tip 108 withdraws the tissue sample and at least a portion of the dissociation fluid at about 10-500 milliliters/second.

In step 412, the dissociation device returns each of the pipette tips 108 to the bottom of each of the wells 110 to form a contact with an angled bottom surface 130 of each of the wells 110.

As described herein, the angled bottom surface 130 of the well 110 is angled with respect to the pipette tip 108. In alternate examples, the pipette tip 108 is angled with respect to either a lower well end 126 or angled bottom surface 130 of the well 110. In general, the lower well end 126 of a well 110 that does not have angled bottom surface 130 will be slightly rounded. However, in some exemplary embodiments lower well end 126 of a well 110 may be flat. The angle of the angled bottom surface 130 of the well 110 in examples may range from about 20 degrees to 45 degrees. The bottom surface of the pipette tips 108 may be rounded.

The positive pressure that the dissociation device provides to the pipette tips 108 is strong enough to provide agitation to the tissue samples to assist with breaking down the tissue samples but gentle enough to not disrupt the cellular structure.

In method 400, at step 402, the pipette tip 108 injects the tissue samples onto the angled bottom surface 130 of each of the wells. The pipette tip 108 dispels the tissue sample from the pipette tip 108 by pump 202 applying positive pressure to the pipette tip 108. For example, the valve controllers 214 may configure any valve actuation to cause the pump 202 to provide an expulsion force (e.g., positive pressure) to any or all of the pipette tips 108. In an example, the outward expulsion may be performed at about 400-600 milliliters/second.

In step 414, the dissociation device twists or rotates each of the pipette tips 108 in a circular motion. The pipette tips 108 may be twisted by any type of twisting force provided by the dissociation device. For example, the pipette tip adaptor 106 may have motorized components that twist the pipette tips 108 in a clockwise and/or counterclockwise direction such as, for example, a plurality of bidirectional stepper motors or a single bidirectional stepper motor linked to a plurality of gears associated with each of the pipette tips 108. Other types of motors that are able to apply a rotational force to each of the plurality of pipette tips 108 are specifically contemplated within the scope of the disclosure. In another example, the wells 110 are twisted by movement of the dissociation well plate 104. For example, the pipette tips 108 may be twisted by from about 90 degrees to about 360 degrees. The twisting action of the pipette tips 108 provides a shearing action to help break down the tissue samples while maintaining the cellular structure.

As described herein, the pipette tips 108 or the angled bottom surface 130 of the well 110 may be serrated (e.g., scalloped, ridged, bubbled, pitted, and the like) or roughened. In certain examples, both the pipette tips 108 and the angled bottom surface 130 may be serrated or roughened. For example, angled bottom surface 130 may be rippled (e.g., similar to a washboard) from the top of the angle to the bottom of the angled to facilitate tissue dissociation when pipette tip 108 is lowered into contact with angled bottom surface 130 in step 412. In certain examples, the serrations or scallops are cut into the surface of the distal pipette ends 122 of pipette tips 108 or onto the angled bottom surface 130 of wells 110 to create a jagged, rough, or uneven surface. In other examples, a rounded material, such as glass beads or other material, may be adhered to the surface of the pipette tip 108. In other examples, the serrations are created with grooves cut into the angled bottom surface 130 of the well 110 to create an uneven surface. In other examples, an uneven material, such as glass beads or other material is adhered to the angled bottom surface 130 of the well 110. When the pipette tip 108 is raised, lowered, and twisted, as described in method 400, the tissue sample may be further broken down. For certain tissue samples, the surface should only include rounded, uneven surfaces that do not have sharp edges. Sharp edges may damage certain cells or nuclei, so rounded glass beads or other rounded materials may be used to make the surface uneven but not jagged or sharp in these embodiments.

In some examples, dots or other roughening material may be located on the pipette tip 108 or the angled bottom surface 130 may be 0.1 mm to 1 mm in diameter.

The pipette tips 108 may be twisted any number of times and left in the well 110 for any suitable amount of time. In each cycle, the tissue sample and fluids may be suctioned into the pipette tip 108, expelled into the well, and twisted or rotated any suitable number of times.

For example, in a single cycle for a fresh tissue sample, the tissue sample may be suctioned and expelled between about 10 and 40 times. In an example for a frozen tissue sample, the tissue sample may be suctioned, expelled, and twisted between about 10 to 25 times. Any suitable number of steps may be used in each cycle based on the type of tissue sample, the dissociation fluids used, and the configuration of the pipette tips 108 and the wells 110.

In steps 318 and 418, the dissociation device repeats the process as required to isolate single-cell or single-nuclei samples. For example, in a single cycle for a fresh tissue sample or for frozen tissue samples, the dissociation device may perform between one and ten, or between five or ten of the cycles. The dissociation device may allow the tissue sample to remain undisturbed in the well for about 10 seconds to about 10 minutes between cycles. Any suitable number of cycles may be used in each process based on the type of tissue sample, the dissociation fluids used, and the configuration of the pipette tips 108 and the wells 110.

At any point in the methods 300 or 400, the pipette tips 108 may be exchanged for fresh pipette tips. For example, after step 312 or step 416, when the tissue sample is returned to the well 110, the pipette tip adaptor 106 or other component may remove the pipette tip 108 from the well 110. A new or clean pipette tip 108 may be affixed to the pipette tip adaptor 106, such as by a friction fitting. The new pipette tip 108 may then be returned to the well 110 to continue the methods 300 or 400.

When single-cell or single-nuclei samples are isolated, the samples may be removed from the wells 110 and used by the lab technician for any suitable purpose, such as for sequencing of the sample.

The dissociation device provides a consistently high yield as compared to manual systems. In an example experiment isolating adult mouse cerebellum nuclei, manual processes obtained approximately 120,000 nuclei per mm³ of tissue sample, while the technology using methods described herein recovered approximately 140,000 nuclei per mm³ of tissue sample.

FIG. 8 is an illustration of DAPI peak results from a conventional device and from the dissociation device as described herein.

After dissociation, the isolated single-cells or single-nuclei may be sorted by a fluorescence-activated cell sorting (“FACS”) process. FACS is a specialized type of flow cytometry that provides a method for sorting a heterogeneous mixture of biological cells into two or more containers, one cell at a time, based upon the specific light scattering and fluorescent characteristics of each cell.

As illustrated, when a DAPI stain is applied to the nuclei, a fluorescence peak is identified. The size of the peak indicates the number of nuclei in the sample. As illustrated, the conventional automated device produced a peak of approximately 1,800 nuclei. The dissociation device of the present disclosure produced a peak of approximately 9,000 nuclei. The dissociation device of the present disclosure consistently produces a greater number of nuclei per sample than other conventional devices.

FIG. 9 is a box and whisker plot of transcripts per nucleus of manual and device dissociated samples.

The box and whisker plot graphically demonstrates the locality, spread and skewness groups of numerical data through their quartiles. The results of two manual dissociation processes and the results of a dissociation with the dissociation device (the “device dissociation”) described herein are illustrated. The boxes illustrated in the box and whisker plot represent the upper quartile and the lower quartile of the results. The line inside the box represents the median result. The upper and lower extremes are illustrated by the horizontal bars above and below the boxes. The outlier results are illustrated with the circles above the upper extreme.

In the illustration of FIG. 9, a number of transcripts per nucleus isolated by the three tests are displayed. The median number of transcripts per nucleus for the dissociation device as disclosed herein is between the results for the two manual dissociations. The upper and lower quartiles are closer to the median in the dissociation with the dissociation device as disclosed herein than in the manual dissociations. Based on the results displayed on the plot, the dissociation with the dissociation device as disclosed herein is substantially equivalent to the results of the manual dissociations.

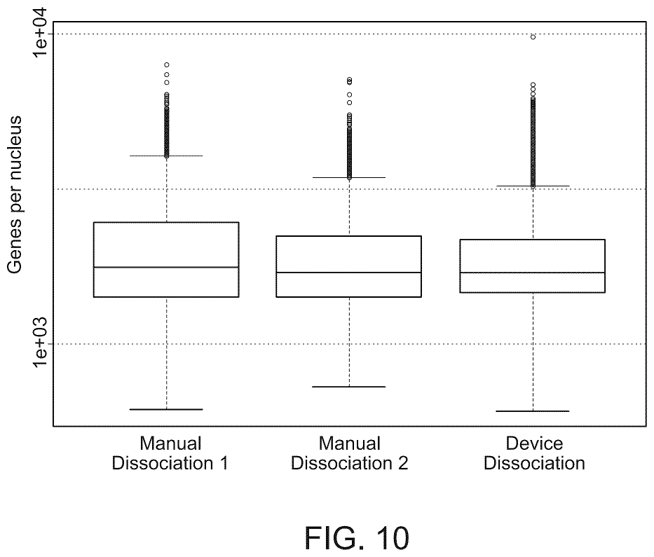

FIG. 10 is a box and whisker plot of genes per nucleus of manual dissociated samples and samples dissociated by the device disclosed herein. In the illustration of FIG. 10, a number of genes per nucleus isolated by the three tests are displayed. The median number of genes per nucleus for the dissociation device are substantially the same as the results for the two manual dissociations. The upper and lower quartiles are closer to the median in the dissociation with the dissociation device of the present disclosure than in the manual dissociations. Based on the results displayed on the plot, the dissociation with the dissociation device as disclosed herein is substantially equivalent to the results of the manual dissociations.

What is claimed is:

1. A tissue dissociation device comprising:
a tissue dissociation plate, said tissue dissociation plate comprising:
a rigid upper surface, and
a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface,
wherein the angled bottom surface comprises a roughened surface internal to each of the plurality of wells.

2. The tissue dissociation device of claim 1, wherein the angled bottom surface has an angle between 10 degrees and 40 degrees between the lower side or the upper side and the circumferential wall.

3. The tissue dissociation device of claim 1, wherein the roughened surface includes one or more ridges, grooves, scallops, bumps, or pits.

4. The tissue dissociation device of claim 3, wherein the one or more ridges, grooves, or scallops substantially span a horizontal diameter of each of the plurality of wells.

5. The tissue dissociation device of claim 3, wherein the one or more ridges, grooves, or scallops are arrayed in a corresponding number of horizontal rows which are substantially equally spaced apart from the lower side to the upper side of the angled bottom surface, or the one or more bumps or pits are substantially evenly spaced across the roughened surface internal to each of the plurality of wells.

6. The tissue dissociation device of claim 4, wherein the one or more ridges, grooves, or scallops each include one or more channels which form breaks in each of the corresponding number of horizontal rows.

7. The tissue dissociation device of claim 1, wherein the upper opening has a diameter between 8 mm to 20 mm, and the circumferential wall has a height of 20 mm to 60 mm.

8. The tissue dissociation device of claim 1, wherein the roughened surface is configured to dissociate a tissue sample into single cells or single nuclei by applying a shearing friction to the tissue sample when the tissue sample is introduced via a pipette tip having a distal tip that is not parallel to the angled bottom surface.

9. A kit to isolate cells and/or single nuclei in a tissue sample, comprising:
a tissue dissociation plate comprising:
a rigid upper surface and a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface,
wherein the angled bottom surface comprises a first roughened surface internal to each of the plurality of wells; and
one or more single cell isolation buffers or single nuclei isolation buffers.

10. The kit of claim 9, further comprising one or more pipette tips each having a distal pipette tip that includes a second roughened surface.

11. The kit of claim 10, wherein the first roughened surface and/or the second roughened surface includes one or more ridges, grooves, scallops, bumps, or pits.

12. The kit of claim 9, wherein the one or more single cell or nuclei isolation buffers include Buffer 1, Buffer 2 or Buffer 3, wherein Buffer 1 comprises Na₂SO₄ (5.83 g), K₂SO₄ (2.615 g), Glucose (0.905 g), HEPES (1.2 g), 1M MgCl₂ (2.5 mL) and ddH₂O added to 500 mL, Buffer 2 comprises Buffer 1 (15 mL), 1% Kollidon (0.150 g), 1% TX-100 (150 μl), and 10% BSA (15 μl), and Buffer 3 comprises Nuclei EZ lysis buffer (Sigma N3408-200ML).

13. The tissue dissociation device of claim 1, further comprising:
at least one pump having one or more pump inlets and one or more pump outlets;
one or more perfusion manifolds having a manifold inlet in fluid connection with one of the one or more pump outlets and two or more manifold outlets in fluid connection with one or more pipette tips via a corresponding number of fluid tubes;
a valve controller configured to regulate fluid flow through the corresponding number of fluid tubes;
a manifold configured to reversibly receive the one or more pipette tips on a lower side and the corresponding number of fluid tubes on an upper side, wherein the manifold is configured to raise, lower, and twist the one or more pipette tips; and
a controller configured to regulate the pump and cause the manifold to perform a sequence of steps to deliver a fluid to each of the plurality of wells via the one or more pipette tips.

14. The tissue dissociation device of claim 13, wherein the tissue dissociation well plate supports an array of 6, 12, 24, 48, 96, 384 or 1536 wells.

15. The tissue dissociation device of claim 13, further comprising an adaptor affixed to the lower side of the manifold configured to reversibly receive the one or more pipette tips.

16. The tissue dissociation device of claim 15, wherein the adaptor further comprises a motor to twist each of the one or more pipette tips in either a clockwise or counterclockwise direction.

17. A method to dissociate tissue samples, comprising:
(i) delivering tissue samples to a plurality of wells arrayed on a tissue dissociation well plate, each tissue dissociation well plate comprising:
a rigid upper surface and a plurality of wells each having an upper opening, a circumferential wall, and an angled bottom surface having a lower side and an upper side, wherein the upper opening of each of the plurality of wells is in the rigid upper surface and the circumferential wall extends between the upper opening and the angled bottom surface and a portion of the circumferential wall adjoining the upper side of the angled bottom surface is shorter than a portion of the circumferential wall adjoining the lower side of the angled bottom surface,
wherein the angled bottom surface comprises a roughened surface internal to each of the plurality of wells;
(ii) delivering one or more single cell isolation buffers or single nuclei isolation buffers to each of the plurality of wells via a plurality of pipette tips;
(iii) withdrawing, via each of the plurality of pipette tips, the tissue samples from each of the plurality of wells via a suction on each of the plurality of pipette tips;
(iv) returning each of the plurality of pipette tips to the bottom of each of the plurality of wells to form a contact with the angled bottom surface of each of the plurality of wells;
(v) injecting the tissue samples onto the angled bottom surface of each of the plurality of wells; and
(vi) twisting each of the plurality of pipette tips in a circular motion.

18. The method to dissociate tissue samples of claim 17, wherein the roughened surface includes one or more ridges, grooves, scallops, bumps, or pits.

19. The method to dissociate tissue samples of claim 18, wherein the one or more ridges, grooves, or scallops substantially span a horizontal diameter of each of the plurality of wells and are arrayed in a corresponding number of horizontal rows which are substantially equally spaced apart from a lower end to an upper end of the angled bottom surface, or wherein the one or more bumps or pits are substantially evenly spaced across the roughened surface internal to each of the plurality of wells.

20. The method to dissociate tissue samples of claim 17, further comprising dissociating a tissue sample into single cells or single nuclei by applying a shearing friction to the tissue sample when the tissue sample is introduced via a pipette tip having a distal tip which is not parallel to the angled bottom surface.
